(19) United States
(12) Plant Patent Application Publication
(10) Pub. No.: US 2017/0339815 P1
(43) Pub. Date: Nov. 23, 2017

(54) Phalaenopsis Orchid Plant Named 'PHALFMODHI'

(50) ;

(71) Applicant: ANTHURA B.V., Bleiswijk (NL)

(72) Inventor: Martinus Nicolaas Gerardus Van Swieten, Utrecht (NL)

(21) Appl. No.: 14/999,515

(22) Filed: May 18, 2016

(51) Int. Cl. A01H 5/02

(52) U.S. Cl. CPC A01H 5/02
USPC PLT/311

(57) ABSTRACT

A new and distinct variety of Phalaenopsis plant named ‘PHALFMODHI’, particularly characterized by having yellow flowers, 1 to 3 peduncles that are long and moderate, leaves that are oblong, and is propagated by meristem tissue culture, is disclosed.

Genus and species: Phalaenopsis hybrid.

Variety denomination: ‘PHALFMODHI’.

### BACKGROUND OF THE NEW PLANT

The present invention relates to a new and distinct cultivar of Phalaenopsis plant, botanically known as Phalaenopsis hybrid of the Orchidaceae family, commonly referred to as moth orchid, and hereinafter referred to by the variety name ‘PHALFMODHI’.

The new Phalaenopsis plant is a product of a planned breeding program conducted by the inventor in Bleiswijk, The Netherlands. The objective of this breeding program was to create a new Phalaenopsis plant with numerous attractive and unique yellow colored flowers, suitable for potted plant production.

The new Phalaenopsis plant ‘PHALFMODHI’ is a result of cross-pollination made by the inventor in August 2006 in Bleiswijk, The Netherlands of the proprietary female, or seed parent, Phalaenopsis hybrid ‘21368-0002’ (unpatented) with the proprietary male, or pollen parent, Phalaenopsis hybrid ‘01-1630’ (unpatented).

The new Phalaenopsis was selected by the inventor as a single plant within the progeny of the stated cross-pollination in a controlled greenhouse in Bleiswijk, The Netherlands in August 2009. Asexual reproduction of the new Phalaenopsis plant by meristem tissue culture since 2012 in Bleiswijk, The Netherlands, has demonstrated that the new variety reproduces true to type with all of the characteristics, as herein described, firmly fixed and retained through successive generations.

Plant Breeder's Rights for this variety have been applied for in Europe on Sep. 24, 2015. ‘PHALFMODHI’ has not been made publicly available or sold anywhere in the world more than one year print to the filing date of this application.

### SUMMARY OF THE INVENTION

The following are the most outstanding and distinguishing characteristics of this new cultivar when grown under normal horticultural practices in Bleiswijk, The Netherlands and can be used to distinguish ‘PHALFMODHI’ as a new and distinct variety of Phalaenopsis plant.
1) Yellow flowers;
2) 1 to 3 peduncles;
3) Peduncle is long and moderate; and
4) Shape of the leaf is oblong.

### DESCRIPTION OF THE PHOTOGRAPHS

This new Phalaenopsis plant is illustrated by the accompanying photographs which show the overall plant habit including blooms and foliage of the plant; the colors shown are as true as can be reasonably obtained by conventional photographic procedures. The photographs were taken in a greenhouse in Bleiswijk, The Netherlands, from 50-week old plants in January 2016. Colors in the photographs may differ from the color values cited in the detailed botanical description, which accurately describe the actual colors of the new variety.

FIG. 1 shows the overall plant habit, including blooms and foliage of ‘PHALFMODHI’.

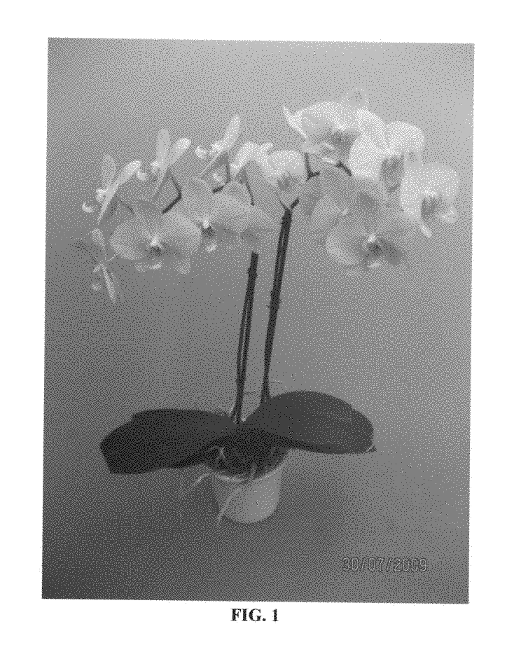

FIG. 2 shows a close-up of a flower of ‘PHALFMODHI’.

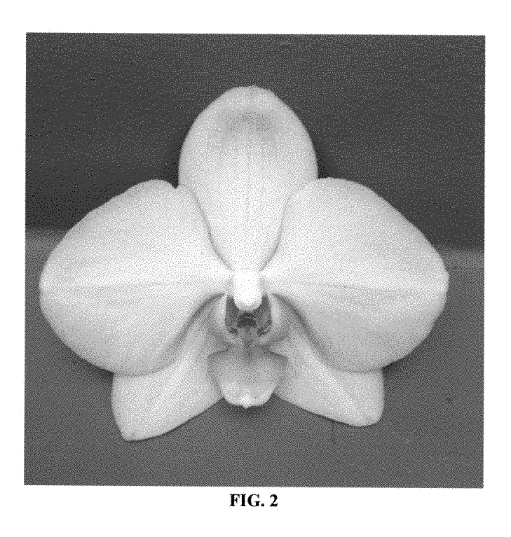

FIG. 3 shows a close-up of the leaves of ‘PHALFMODHI’.

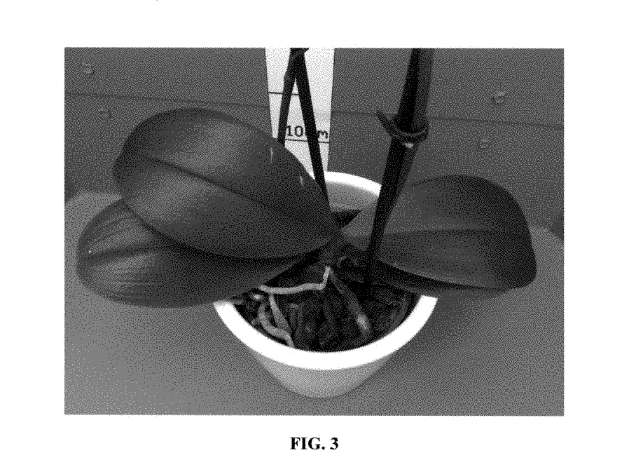

### DESCRIPTION OF THE NEW VARIETY

The following detailed description sets forth the distinctive characteristics of ‘PHALFMODHI’ . Plants of the new Phalaenopsis have not been observed under all possible environmental conditions. The phenotype may vary somewhat with variations in environment such as temperature, light intensity and day length, without, however, any variance in genotype. The chart used in the identification of colors described herein is The R.H.S. Colour Chart of The Royal Horticultural Society, London, England, 2015 edition, except where general color terms of ordinary significance are used. The color values were determined under 4000-6000 lux natural light in a greenhouse in Bleiswijk, The Netherlands. Observations and measurements were made in January 2016 on 50-week old plants which were planted from a nursery tray in 12 centimeter (diameter) pots and grown in a greenhouse between 27° C. to 29° C. for 30 weeks, continued by a cooling period of 8 weeks between 18° C. to 20° C. and 12 weeks in a greenhouse of 21° C.

### DETAILED BOTANICAL DESCRIPTION

Classification:
   Family.—Orchidaceae.
   Botanical.—Phalaenopsis hybrid.
   Common name.—Phalaenopsis.
   Variety name.—‘PHALFMODHI’.
Parentage:
   Female parent.—Phalaenopsis cultivar ‘21368-0002’ (unpatented).
   Male parent.—Phalaenopsis cultivar ‘01-1630’ (unpatented).
Propagation:
   Type.—Meristem tissue culture.
Roots:
   Root description.—Grey-green colored roots with lightly branching lateral roots having light green colored root tips.
Plant:
   Commercial crop time to flowering.—Approximately 48 to 50 weeks from a rooted cutting to finish in a 12 cm pot.
   Growth habit of peduncle.—Standard, green leaves, raceme to panicle.
   Height (from soil level to top of inflorescence).—Approximately 45.0 cm to 55.0 cm.
   Width (measured from leaf tips).—About 29.0 cm to 32.0 cm.
   Vigor.—Moderate.
Leaves:
   Mature leaves.—Quantity per plant: 6 to 7 leaves are produced before flowering Length (fully expanded): 15.0 cm to 17.0 cm Width: 8.0 cm to 9.0 cm Shape: Oblong Base shape: Moderately elongated Apex: Unequal obtuse Leaf blade angle with the petiole (measured from the horizontal position): Between 25 degrees and 45 degrees Leaf margin: Entire Color: Upper surface: RHS 147A Lower surface: RHS 146A with edge RHS 147A Texture: Slightly rough Thickness: 2.5 mm to 2.8 mm Venation: Pattern: Parallel Color of the midvein: Upper surface: RHS 147A Lower surface: RHS 146A.
Peduncle:
   Quantity per plant.—1 to 3.
   Number of flowers per peduncle.—8 to 11.
   Length.—53.0 cm to 58.0 cm.
   Diameter.—4.4 mm to 4.9 mm.
   Strength.—Moderate.
   Aspect.—Upright to slightly pendant.
   Texture.—Smooth.
   Color.—Green (RHS 146B) with a touch of brown (RHS 200D).
   Internode length.—40.0 mm to 50.0 mm.
   Callosities.—None.
Inflorescence description:
   Appearance.—Uptight to slightly pendant, raceme to panicle inflorescence with bilaterally symmetrical flowers that open in succession beginning with the lowermost flower.
   Inflorescence size.—Height (from base to tip): 200.0 mm to 220.0 mm Diameter: 3.6 mm to 3.8 mm.
   Flowering time.—First flowers can be expected 10 to 11 months after planting in a 12 cm (diameter) pot.
   Flower.—Height: 92.0 mm to 97.0 mm Diameter: 90.0 mm to 95.0 mm. Depth of lip: 19.0 mm to 21.0 mm
   Flower longevity.—On the plant: 13 to 21 weeks.
   Fragrance.—Absent.
   Flower bud.—Average size: Large Length: 25.0 mm to 27.0 mm Width: 21.0 mm to 23.0 mm Shape: Egg shaped Color: Green (RHS 145A).
   Petals.—Arrangement: Open/free Shape: Semi-circular Apex: Emarginated asymmetric Margin: Entire Length (from base to tip): 43.0 mm to 45.0 mm Width: 49.0 mm to 51.0 mm Color (when fully opened): Upper surface: Basic color: Yellow-green (RHS 1D) Over color: Absent Lower surface: Basic color: Yellow-green (RHS 1D) Over color: Absent.
   Dorsal sepal.—Shape: Elliptic Apex: Emarginated symmetric Margin: Entire Length (from base to tip): 47.0 mm to 49.0 mm Width: 34.0 mm to 36.0 mm Color (when fully opened): Upper surface: Basic color: Yellow-green (RHS 1D) Over color: Absent Lower surface: Basic color: Yellow-green (RHS 1D) Over color: Green-yellow in the middle (RHS 150C).
   Lateral sepals.—Shape: Ovate Apex: Obtuse symmetric Margin: Entire Length (from base to tip): 46.0 mm to 48.0 mm Width: 30.0 mm to 32.0 mm Color (when fully opened): Upper surface: Basic color: Yellow (RHS 1D) Over color: Touch of greyed-orange at the base (RHS 172B) Lower surface: Basic color: Yellow (RHS 1D) Over color: Green-yellow in the middle (RHS N144D).
   Labellum (lip).—Margin: Entire Whiskers: Present Length of whiskers: 9.0 mm to 11.0 mm Color of whiskers: White (RHS 155C) with slightly yellow tips (RHS 2D) Pubescence on the lip: Absent.
   Lateral lobe.—Shape: Weakly spatulate Length (from base to tip): 20.0 mm to 22.0 mm Width: 9.0 mm to 11.0 mm Color: White (RHS 155C) and yellow-orange on one side (RHS 17A) with orange-red spots towards the base (RHS N34)
   Apical lobe.—Shape: Trullate Length: 21.0 mm to 23.0 mm Width: 19.0 mm to 21.0 mm Color: Greyed-orange at the base (RHS 173B); yellow (RHS 12A) and white (RHS 155C) towards the whiskers.
   Callus.—Average size: Medium Height: 0.6 cm to 0.7 cm Length: 0.5 cm to 0.6 cm Width: 0.5 cm to 0.6 cm Color: Yellow (RHS 17A) with greyed-red spots (RHS N34).
   Reproductive organs.—Column: Length: 11.0 mm to 13.0 mm Diameter: 5.9 mm Color: White (RHS 155C).
   Pollinia.—Quantity: 2 Diameter: 0.9 mm to 1.0 mm Color: Yellow-orange (RHS 17A)
   Ovary.—Length: 9.0 mm to 11.0 mm Diameter: 2.4 mm to 2.6 mm.
   Pedicel.—Length: 37.0 mm to 39.0 mm Diameter: 3.3 mm to 3.6 mm Color: Green at the base (RHS 146C) and lighter green towards the flower (RHS 145B to 145C)
Disease, pest, and stress resistance: No specific resistance or susceptibility observed to pathogens and pests common to Phalaenopsis.
Fruit and seeds: Fruit and seed development has not been observed on plants of the new Phalaenopsis.

### COMPARISON WITH PARENTAL AND COMMERCIAL VARIETIES

‘PHALFMODHI’ differs from female parent plant ‘21368-0002’ (unpatented) in that ‘PHALFMODHI’ has evenly colored flowers and a medium-sized callus, whereas ‘21368-0002’ has dotted flowers and a very small callus. Additionally, ‘PHALFMODHI’ has wider flowers and longer lip whiskers than ‘21368-0002’.

‘PHALFMODHI’ differs from the male parent ‘01-1630’ (unpatented) in that ‘PHALFMODHI’ has a weakly spatulate shaped lateral lobe, whereas ‘01-1630’ has a spatulate shaped lateral lobe. Additionally, ‘PHALFMODHI’ has narrower flowers and shorter lip whiskers than those of ‘01-1630’.

‘PHALFMODHI’ is most similar to the commercial Phalaenopsis plant named ‘PHALCUZO’ (unpatented). ‘PHALFMODHI’ differs from commercial variety ‘PHALCUZO’ in that ‘PHALFMODHI’ has a yellow and white apical lobe of the lip with a touch of greyed-orange at the base and a callus with very small dots, whereas ‘PHALCUZO’ has a greyed-orange and white apical lobe of the lip with a touch of yellow and a striped callus with larger dots. Additionally, ‘PHALFMODHI’ has a backside of the apical lobe that is yellow at the base, whereas ‘PHACUZO’ has a backside of the apical lobe that is purple/greyed-purple at the base.

I claim:

1. A new and distinct variety of Phalaenopsis plant named ‘PHALFMODHI’, substantially as described and illustrated herein.
